(19) United States
(12) Patent Application Publication
(10) Pub. No.: US 2024/0398727 A1
(43) Pub. Date: Dec. 5, 2024

(54) (S)-5-(1-(3,5-BIS(TRIFLUOROMETHYL)PHENOXY)ETHYL)CYCLOHEXANE-1,3-DIONE (NU-9) IMPROVES THE HEALTH OF DISEASED UPPER MOTOR NEURONS

(71) Applicant: Northwestern University, Evanston, IL (US)

(72) Inventors: Pembe Hande Ozdinler, Evanston, IL (US); Richard Bruce Silverman, Evanston, IL (US)

(21) Appl. No.: 18/694,882

(22) PCT Filed: Sep. 28, 2022
(86) PCT No.: PCT/US22/77195

§ 371 Date: Mar. 22, 2024

Related U.S. Application Data
(60) Provisional application No. 63/261,779, filed on Sep. 28, 2021.

(51) Int. Cl. A61K 31/122; A61K 31/4152; A61K 31/428; A61P 25/02; A61P 25/28; G01N 33/50

(52) U.S. Cl. CPC A61K 31/122; A61K 31/4152; A61K 31/428; A61P 25/02; A61P 25/28; G01N 33/5008

(57) ABSTRACT

Disclosed are compositions and methods for treating amyotrophic lateral sclerosis (ALS) and compositions and methods for improving the health of diseased upper motor neurons. Particularly disclosed are compositions for improving the health of diseased upper motor neurons (UMNs) with additive effects in combination with drugs for treating ALS. The disclosed composition and methods may include or utilize (S)-5-(1-(3,5-bis(trifluoromethyl)phenoxy)ethyl)cyclohexane-1, 3-dione (NU-9), for example, in order to improve the health of diseased UMNs with additive effects in combination with drugs for treating ALS.

### CROSS REFERENCE TO RELATED APPLICATIONS

This application claims benefit of priority to U.S. Patent Application Ser. No. 63/261,779, filed Sep. 28, 2021, the contents of which is incorporated by reference in its entirety.

### STATEMENT REGARDING FEDERALLY SPONSORED RESEARCH OR DEVELOPMENT

This invention was made with government support under 5R01AG061708-02 awarded by the National Institutes of Health. The government has certain rights in the invention.

### BACKGROUND

Amyotrophic lateral sclerosis (ALS) is a disease of the motor neuron circuitry, which has components both in the brain and in the spinal cord. Even though the spinal motor neuron component of ALS is better studied, our understanding of the cortical component and the biology of upper motor neurons (UMNs) is still limited. There has been no preclinical drug screening platform for diseased UMNs, because there has been no interest in finding out whether compounds considered for clinical trials also improved the health of diseased UMNs. There is an urgent need to develop better and translational preclinical assays that include UMN health as a readout, which will expedite drug discovery efforts.

### SUMMARY

The field of the invention relates to compositions and methods for treating amyotrophic lateral sclerosis (ALS) and compositions and methods for improving the health of diseased upper motor neurons. In particular, the field of the invention relates to compositions for improving the health of diseased upper motor neurons (UMNs) with additive effects in combination with drugs for treating ALS. The disclosed composition and methods may include or utilize (S)-5-(1-(3,5-bis(trifluoromethyl)phenoxy)ethyl)cyclohexane-1,3-dione (NU-9), for example, in order to improve the health of diseased UMNs with additive effects in combination with drugs for treating ALS such as riluzole and/or edaravone. (S)-5-(1-(3,5-bis(trifluoromethyl)phenoxy)ethyl)cyclohexane-1,3-dione (NU-9) has a structural formula of

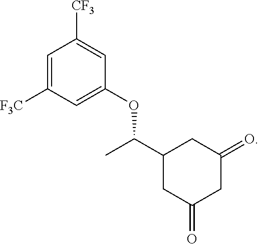 [SMILES: C[C@H](Oc1cc(C(F)(F)F)cc(C(F)(F)F)c1)C1CC(=O)CC(=O)C1]

NU-9 improves UMN axon outgrowth in vitro, better than FDA approved ALS drugs edaravone and riluzole, and when NU-9 is used in combination with riluzole or edaravone, there is a combinatorial effect.

In one aspect of the current disclosure, methods of treating ALS or the symptoms in a subject in need thereof are provided. In some embodiments, the methods comprise: administering an effective amount of (S)-5-(1-(3,5-bis(trifluoromethyl)phenoxy)ethyl)cyclohexane-1,3-dione, or a suitable pharmaceutical salt thereof, to the subject to treat ALS in the subject. In some embodiments, the subject is administered a daily dose of the compound of about 100 mg/kg, 75 mg/kg, 50 mg/kg, 25 mg/kg, 20 mg/kg, 10 mg/kg, 5 mg/kg, 1 mg/kg, 0.5 mg/kg, 0.1 mg/kg, 0.05 mg/kg, 0.01 mg/kg or lower, or within a range bounded by any of these values. In some embodiments, the compound is administered orally. In some embodiments, the methods further comprise administering riluzole, edaravone, or pharmaceutically acceptable salts thereof.

In another aspect of the current disclosure, methods for improving the health of diseased upper motor neurons in a subject in need thereof are provided. In some embodiments, the methods comprise administering an effective amount of (S)-5-(1-(3,5-bis(trifluoromethyl)phenoxy)ethyl)cyclohexane-1,3-dione, or a suitable pharmaceutical salt thereof, to the subject to treat a disease or disorder associated with diseased upper motor neurons. In some embodiments, the disease or disorder is ALS. In some embodiments, the method treats memory loss in the subject. In some embodiments, the subject is administered a daily dose of the compound of about 100 mg/kg, 75 mg/kg, 50 mg/kg, 25 mg/kg, 20 mg/kg, 10 mg/kg, 5 mg/kg, 1 mg/kg, 0.5 mg/kg, 0.1 mg/kg, 0.05 mg/kg, 0.01 mg/kg or lower, or within a range bounded by any of these values. In some embodiments, the compound is administered orally. In some embodiments, the methods further comprise administering riluzole, edaravone, or pharmaceutically acceptable salts thereof.

In another aspect of the current disclosure unit dosage packages are provided. In some embodiments, the unit dosage packages comprise: (i) (S)-5-(1-(3,5-bis(trifluoromethyl)phenoxy)ethyl)cyclohexane-1,3-dione, or a pharmaceutically acceptable salt thereof; and (ii) riluzole, edaravone, or pharmaceutically acceptable salts.

In another aspect of the current disclosure, pharmaceutical compositions are provided. In some embodiments, the pharmaceutical compositions comprise: (i) (S)-5-(1-(3,5-bis(trifluoromethyl)phenoxy)ethyl)cyclohexane-1,3-dione, or a pharmaceutically acceptable salt thereof; (ii) riluzole, edaravone, or pharmaceutically acceptable salts; and (iii) a pharmaceutically acceptable carrier or excipient.

In another aspect of the current disclosure, methods for detecting candidate compounds that that improve health of diseased upper motor neurons are provided. In some embodiments, the methods comprise: (i) culturing diseased upper motor neurons in the presence and absence of a candidate compound; (ii) detecting one or more parameters related to upper motor neuron health in the cells of step (i); (iii) generating a test index by calculating a change in the one or more parameters between the diseased upper motor neurons cultured in the presence and absence of the candidate compound and generating a control index by calculating a change in the one or more parameters between the cells cultured in the presence and absence of a control substance; wherein, if the value of the test index is greater than, or improved, as compared to the value of the control index, then the candidate compound improves the health of diseased upper motor neurons. In some embodiments, the diseased motor neurons become diseased by mSOD1 toxicity and/or TDP-43 pathology. In some embodiments, the one or more parameters between the diseased upper motor neurons cultured in the presence and absence of the candidate compound is axon length or neuronal arborization or branching. In some embodiments, the control substance comprises a serum free medium, riluzole, edaravone, AMX-0035, NU-9, or any combination thereof.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1. SOD Figure: NU-9 enhances axon outgrowth of UMNs that become diseased by misfolded SOD1 toxicity. a Representative images of UMNs in dissociated cell cultures of motor cortex isolated from WT-UeGFP and b hSOD1^{G93A}-UeGFP mice treated with SFM or c-f with 400 nM of NU-9, g 500 nM riluzole, or h 1 μM edaravone for 3 days in vitro. Dots (DAPI) represent other cells in culture, whereas UMNs are identified by their eGFP expression. Scale bars, 25 μm; n=3 biological replicates. i Average length of UMN axons in WT-UeGFP or hSOD1^{G93A}-UeGFP mice treated with 400 nM NU-9, 500 nM riluzole, 1 μM edaravone, AMX-0035 [1 mM 4-Phenylbutyric acid (4-PBA), +100 μM Tauroursodeoxycholic acid, sodium salt (TUDCA)], or combination of NU-9 with riluzole, edaravone, or AMX-0035; mean, SEM, and individual data points shown for n=3 biological replicates. *p<0.05, **p<0.01, ***p<0.001, ****p<0.0001, one-way ANOVA followed by Tukey's post hoc multiple-comparison test. j Percent distribution of UMN based on axon length in WT-UeGFP or hSOD1^{G93A}-UeGFP mice treated with 400 nM NU-9, 500 nM riluzole, 1 μM edaravone, AMX-0035 [1 mM 4-Phenylbutyric acid (4-PBA), +100 μM Tauroursodeoxycholic acid, sodium salt (TUDCA)], or combination of NU-9 with riluzole, edaravone, or AMX-0035. SFM=serum free medium; G=WT-UeGFP; GS=hSOD1G93A-UeGFP.

FIG. 2. TDP Figure: NU-9 enhances axon outgrowth of UMNs that become diseased from TDP-43 pathology. a Representative images of UMNs in dissociated cell cultures of motor cortex isolated from WT-UeGFP and b prpTDP-43^{A315T}-UeGFP mice treated with SFM, c-f 400 nM NU-9, g 500 nM riluzole, or h 1 μM edaravone for 3 days in vitro. Dots (DAPI) represent other cells in culture, whereas UMNs are identified by their eGFP expression. Scale bars, 25 μm; n=3 biological replicates. i Average length of the longest neurite of UMNs from WT-UeGFP or prpTDP-43^{A315T}-UeGFP mice treated with 400 nM NU-9, 500 nM riluzole, 1 μM edaravone, or combination of NU-9 with riluzole or edaravone; mean, SEM, and individual data points shown for n=3 biological replicates. *p<0.05, **p<0.01, one-way ANOVA followed by Tukey's post hoc multiple-comparison test. j Percent distribution of UMNs based on axon length of WT-UeGFP or prpTDP-43^{A315T}-UeGFP mice treated with 400 nM NU-9, 500 nM riluzole, 1 μM edaravone, or combination of NU-9 with riluzole or edaravone. SFM=serum free medium; G=WT-UeGFP; GS=hSOD1G93A-UeGFP.

### DETAILED DESCRIPTION

The present invention is described herein using several definitions, as set forth below and throughout the application.

### Definitions

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

A “subject in need thereof” as utilized herein may refer to a subject in need of treatment for a disease or disorder associated with diseased upper motor neurons. A subject in need thereof may include a subject having a disease or disorder that is characterized by shorter axon lengths and/or extend of neuronal arborization or branching. A “subject in need thereof” as utilized herein may include, but is not limited to a subject in need of treatment of ALS.

The term “subject” may be used interchangeably with the terms “individual” and “patient” and includes human and non-human mammalian subjects.

The disclosed compounds, pharmaceutical compositions, and methods may be utilized to treat and/or prevent diseases and disorders associated with diseased UMNs, such as ALS.

The disclosed compounds may be utilized to improve the health of UMNs. Improved health of UMNs can be charactered by increasing axon length, increasing neuronal arborization or branching, or increasing both axon length and increasing neuronal arborization or branching. As demonstrated in the Examples, NU-9 effectively increasing both axon length and increasing neuronal arborization or branching.

### Methods of Use

In one aspect of the current disclosure, methods of treating ALS or symptoms thereof in a subject in need thereof are provided. The methods comprise administering an effective amount of NU-9, or a suitable pharmaceutical salt thereof, to the subject to treat ALS disease in the subject.

In another aspect of the current disclosure, methods for improving the health of diseased upper motor neurons in a subject in need thereof are provided. The methods comprise administering an effective amount of NU-9, or a suitable pharmaceutical salt thereof, to the subject to improve the health of upper motor neurons in the subject.

The methods of the instant disclosure may further comprise administering at least one other compound to the subject selected from riluzole (2-Amino-6-(trifluoromethoxy)benzothiazole; CAS 1744-22-5), edaravone (2,4-Dihydro-5-methyl-2-phenyl-3H-pyrazol-3-one; CAS 89-25-8), or a pharmaceutically acceptable salt thereof. In some embodiments, riluzole, or a pharmaceutically acceptable salt thereof, is administered to the subject in combination with NU-9. In some embodiments, edaravone, or a pharmaceutically acceptable salt thereof, is administered to the subject in combination with NU-9.

Disclosed are compounds, pharmaceutical compositions comprising the compounds, and methods of using the compounds and pharmaceutical compositions for treating and/or preventing a disease or disorder associated with diseased upper motor neurons in a subject in need thereof. The disclosed compounds may include cyclohexane 1,3-diones, such as NU-9 and pharmaceutically acceptable salts thereof, that improve the health of the upper motor neurons. Suitably, the disclosed compounds may increase axon length and/or neuronal arborization or branching As such, the disclosed compounds and pharmaceutical compositions may be utilized in methods for treating a subject having or at risk for developing a disease or disorder that is associated with diseased upper motor neurons which may be disease and disorders associated with ALS.

The disclosed compounds include cyclohexane 1,3-diones. Cyclohexane 1,3-diones and methods for synthesizing cyclohexane 1,3-diones are disclosed in the art. (See e.g., Zhang et al., “Chiral Cyclohexan 1,3-diones as Inhibitors of Mutant SOD1-Dependent Protein Aggregation for the Treatment of ALS,” ACS Medic. Chem. Lett., 20021, 3, 584-587, the content of which is incorporated herein by reference in its entirety). The disclosed compounds for uses as disclosed herein may include, but are not limited to (S)-5-(1-(3,5-bis(trifluoromethyl)phenoxy)ethyl)cyclohexane-1,3-dione (NU-9).

In some embodiments, the disclosed methods may be performed in order to treat and/or prevent a disease or disorder is selected from, but not limited to, ALS. In some embodiments, the disclosed methods may be performed in order to treat and/or prevent one or more symptoms of a disease or disorder associated with ALS.

The disclosed methods may be performed in order to improve the health of diseased motor neurons in a subject. For example, the disclosed methods may be performed in order to treat and/or prevent ALS in a subject that is associated with improve the health of diseased motor neurons.

In the disclosed methods, the subject may be administered an effective amount of the disclosed compounds in order to treat and/or prevent amyloid beta oligomerization in the subject. In some embodiments of the disclosed methods, the subject is administered a daily dose of the disclosed compounds of about 100 mg/kg, 75 mg/kg, 50 mg/kg, 25 mg/kg, 20 mg/kg, 10 mg/kg, 5 mg/kg, 1 mg/kg, 0.5 mg/kg, 0.1 mg/kg, 0.05 mg/kg, 0.01 mg/kg or lower, or within a range bounded by any of these values.

In the disclosed methods, the compounds and pharmaceutical compositions may be administered to the subject by any suitable route in order to deliver an effective amount of the disclosed compounds to a site in a subject that is exhibiting diseased upper motor neurons or to a site in the subject that is at risk for incurring upper motor neurons, such as the brain of the subject. In some embodiments, the compounds and pharmaceutical compositions are administered through an oral route.

### Pharmaceutical Compositions

In another aspect of the current disclosure, pharmaceutical compositions are provided. In some embodiments, the pharmaceutical compositions comprise: (i) NU-9, or a pharmaceutically acceptable salt thereof, and (ii) a pharmaceutically acceptable carrier or excipient.

In some embodiments, the pharmaceutical compositions combine NU-9 with another compound for use in the treatment of ALS or to improve the health of diseased upper motor neurons. The pharmaceutical compositions may comprise: (i) NU-9, or a pharmaceutically acceptable salt thereof; (ii) riluzole, or a pharmaceutically acceptable salt thereof, or edaravone, or a pharmaceutically acceptable salt thereof; and (iii) a pharmaceutically acceptable carrier or excipient. In some embodiments, the pharmaceutical compositions may comprise: (i) NU-9, or a pharmaceutically acceptable salt thereof; (ii) riluzole, or a pharmaceutically acceptable salt thereof; and (iii) a pharmaceutically acceptable carrier or excipient. The pharmaceutical compositions may comprise: (i) NU-9, or a pharmaceutically acceptable salt thereof; (ii) edaravone, or a pharmaceutically acceptable salt thereof; and (iii) a pharmaceutically acceptable carrier or excipient.

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures.

The compounds for use according to the methods of disclosed herein may be administered as a single compound or a combination of compounds. For example, a compound that improves the health of diseased motor neurons may be administered as a single compound or in combination with another compound that improves the health of diseased motor neurons or that has a different pharmacological activity.

As indicated above, pharmaceutically acceptable salts of the compounds are contemplated and also may be utilized in the disclosed methods. The term “pharmaceutically acceptable salt” as used herein, refers to salts of the compounds, which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-. 1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, α-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

The particular counter-ion forming a part of any salt of a compound disclosed herein is may not be critical to the activity of the compound, so long as the salt as a whole is pharmacologically acceptable and as long as the counter-ion does not contribute undesired qualities to the salt as a whole. Undesired qualities may include undesirably solubility or toxicity.

Pharmaceutically acceptable esters and amides of the compounds can also be employed in the compositions and methods disclosed herein. Examples of suitable esters include alkyl, aryl, and arylalkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like.

In addition, the methods disclosed herein may be practiced using solvate forms of the compounds or salts, esters, and/or amides, thereof. Solvate forms may include ethanol solvates, hydrates, and the like.

The pharmaceutical compositions may be utilized in methods of treating a disease or disorder associated with diseased upper motor neurons or ALS. As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

As used herein the term “effective amount” refers to the amount or dose of the compound, upon single or multiple dose administration to the subject, which provides the desired effect in the subject under diagnosis or treatment. The disclosed methods may include administering an effective amount of the disclosed compounds (e.g., as present in a pharmaceutical composition) for treating a disease or disorder associated with biological activity of amyloid beta. An effective amount may improve the health of upper motor neurons. In some embodiments, an effective amount may increase axon length and/or neuronal arborization or branching.

An effective amount can be readily determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

A typical daily dose may contain from about 0.01 mg/kg to about 100 mg/kg (such as from about 0.05 mg/kg to about 50 mg/kg and/or from about 0.1 mg/kg to about 25 mg/kg) of each compound used in the present method of treatment.

Compositions can be formulated in a unit dosage form, each dosage containing from about 1 to about 1000 mg of each compound individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

Oral administration is an illustrative route of administering the compounds employed in the compositions and methods disclosed herein. Other illustrative routes of administration include transdermal, percutaneous, intravenous, intramuscular, intranasal, buccal, intrathecal, intracerebral, or intrarectal routes. The route of administration may be varied in any way, limited by the physical properties of the compounds being employed and the convenience of the subject and the caregiver.

As one skilled in the art will appreciate, suitable formulations include those that are suitable for more than one route of administration. For example, the formulation can be one that is suitable for both intrathecal and intracerebral administration. Alternatively, suitable formulations include those that are suitable for only one route of administration as well as those that are suitable for one or more routes of administration, but not suitable for one or more other routes of administration. For example, the formulation can be one that is suitable for oral, transdermal, percutaneous, intravenous, intramuscular, intranasal, buccal, and/or intrathecal administration but not suitable for intracerebral administration.

The inert ingredients and manner of formulation of the pharmaceutical compositions are conventional. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches, and suspensions. In general, compositions contain from about 0.5% to about 50% of the compound in total, depending on the desired doses and the type of composition to be used. The amount of the compound, however, is best defined as the “effective amount”, that is, the amount of the compound which provides the desired dose to the patient in need of such treatment. The activity of the compounds employed in the compositions and methods disclosed herein are not believed to depend greatly on the nature of the composition, and, therefore, the compositions can be chosen and formulated primarily or solely for convenience and economy.

Capsules are prepared by mixing the compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders.

Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects.

A lubricant can be used in the tablet formulation to prevent the tablet and punches from sticking in the die. The lubricant can be chosen from such slippery solids as talc, magnesium and calcium stearate, stearic acid, and hydrogenated vegetable oils.

Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

Transdermal patches can also be used to deliver the compounds. Transdermal patches can include a resinous composition in which the compound will dissolve or partially dissolve; and a film which protects the composition, and which holds the resinous composition in contact with the skin. Other, more complicated patch compositions can also be used, such as those having a membrane pierced with a plurality of pores through which the drugs are pumped by osmotic action.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

### Unit Dosage Packages

In another aspect of the current disclosure, unit dosage packages are provided. Unit dosage packages comprise a first unit dosage including a first drug, such as NU-9 or a pharmaceutically acceptable salt thereof. Unit dosage packages may also comprise a second unit dosage comprising a second drug. The unit dosage package may comprise a container or label indicating the name, strength, control number, expiration date, administration instructions, or any combination thereof for the one or more drugs in the unit dosage package.

In some embodiments, the unit dosage packages comprise NU-9, or a pharmaceutically acceptable salt thereof, for treating ALS or improving the health of diseased upper motor neurons. The unit dosage packages comprise: (i) NU-9, or a pharmaceutically acceptable salt thereof; and (ii) riluzole, or a pharmaceutically acceptable salt thereof, or edaravone, or a pharmaceutically acceptable salt thereof. In some embodiments, the unit dosage packages comprise: (i) NU-9, or a pharmaceutically acceptable salt thereof; and (ii) riluzole, or a pharmaceutically acceptable salt thereof. In some embodiments, the unit dosage packages comprise: (i) NU-9, or a pharmaceutically acceptable salt thereof; and (ii) edaravone, or a pharmaceutically acceptable salt thereof.

### Methods of Detecting a Candidate Compound

In another aspect of the current disclosure, methods of detecting a candidate compound that improve the health of diseased upper motor neurons. In some embodiments, the methods comprise: (i) culturing diseased upper motor neurons in the presence and absence of a candidate compound, or a pharmaceutically acceptable salt thereof; (ii) detecting one or more parameters related to upper motor neuron health in the cells of step (i); (iii) generating a test index by calculating a change in the one or more parameters between the diseased upper motor neurons cultured in the presence and absence of the candidate compound and generating a control index by calculating a change in the one or more parameters between the cells cultured in the presence and absence of a control substance; wherein, if the value of the test index is greater than, or improved, as compared to the value of the control index, then the candidate compound improves the health of diseased upper motor neurons.

As used herein, use of the phrase “improved as compared to the value of the control index” refers to the test index having a greater value than the control index in a situation where the greater value is associated with a beneficial effect or a reduced value than the control index in a situation where the reduced value is associated with a beneficial effect. The phrase reflects that some values associated with a beneficial effect may be increased in the presence of the control substance or decreased in the presence of the control substance.

The control substance may comprise a substance that is not expected to improve the health of diseased upper motor neurons. An exemplary control substance would not be expected to improve the health of diseased upper motor neurons is serum free medium, which was utilized in the Examples. The control substance may comprise a substance that does or is suspected to improve the health of diseased upper motor neurons. Exemplary control substances that do or would be suspected to improve the health of diseased upper motor neurons is riluzole, edaravone, AMX-0035 (a co-formulation of two active pharmaceutical ingredients (APIs), sodium phenylbutyrate (PB) and taurursodiol (TURSO), NU-9, and combinations thereof, which were utilized in the Examples. In some embodiments, the control substance may comprise any of the foregoing.

In some embodiments, the one or more parameters related to upper motor neuron health includes axon length and/or neuronal arborization or branching. The length of axons may be determined through various techniques known in the art, e.g., fluorescent microscopy, or other means known in the art to identify, locate, or characterize the length of the axon. The 1 neuronal arborization or branching may be determined through various techniques known in the art, e.g., fluorescent microscopy, or other means known in the art to identify, locate, or characterize neuronal arborization or branching. Exemplary methods for determining axon length or neuronal arborization or branching are provided in the Examples.

The diseased upper motor neurons may become diseased by mSOD1 toxicity and/or TDP-43 pathology. Misfolded SOD1 toxicity and TDP-43 pathology represent two distinct, and mostly nonoverlapping, causes of ALS. Therefore, being able to identify a compound that improves the health and stability of upper motor neurons that become diseased due to these two different causes would have implications for a broad spectrum of patients. mSOD1 toxicity refers to disease associated with misfolded superoxide dismutase protein (mSOD1). TDP-43 pathology refers to upper motor neurons that have inclusions in transactive response DNA-binding protein 43 (TDP-43) that may result in defects in their mitochondria and endoplasmic reticulum. The diseased upper motor neurons may be prepared so as to express a fluorescent protein, such as enhanced green fluorescent protein (eGFP). Other fluorescent proteins may also be utilized, and numerous fluorescent proteins and expression systems are known in the art. Exemplary methods for preparing and utilizing diseased motor neurons are provided in the Examples.

### EXAMPLES

The following Examples are illustrative and should not be interpreted to limit the scope of the claimed subject matter.

### Example. NU-9 Eliminates Degeneration of Upper Motor Neurons Diseased by mSOD1 Toxicity and TDP-43 Pathology In Vitro

Amyotrophic lateral sclerosis (ALS) is a disease of the motor neuron circuitry, which has components both in the brain and in the spinal cord. Even though the spinal motor neuron component of ALS is better studied, our understanding of the cortical component and the biology of upper motor neurons (UMNs) is still limited. There has been no preclinical drug screening platform for diseased UMNs, because there has been no interest in finding out whether compounds considered for clinical trials also improved the health of diseased UMNs. There is an urgent need to develop better and translational preclinical assays that include UMN health as a readout, which will expedite drug discovery efforts. Here we describe a novel in vitro drug verification platform, in which our goal is not to screen thousands of drugs, but to investigate whether the compounds of interest also improve the health and stability of UMNs. e show that NU-9 improves UMN axon outgrowth in vitro, better than FDA approved ALS drugs edaravone and riluzole.

The only two drugs that have been approved by the FDA to treat ALS are riluzole, approved in 1995, and edaravone, approved in 2017; the latter works as a free radical scavenger and has been previously prescribed for stroke patients (Yoshino and Kimura 2006, Ito, Wate et al. 2008, Yoshida, Kwon et al. 2008, Yoshida, Kwon et al. 2008, Ikeda and Iwasaki 2015, Writing and Edaravone 2017). The ability of edaravone to improve UMN health has not been tested, and its efficacy has been studied only with the SOD1, and not on the TDP-43, mouse model. Limited information is available on the cellular events that contribute to improved motor neuron survival (Ikeda and Iwasaki 2015, Homma, Kobayashi et al. 2019). Riluzole was approved prior to the development of hSOD1^{G93A} mice, and it works mainly on astrocytes to reduce astrogliosis mediated toxicity (Bellingham 2011). Because riluzole failed to improve the longevity of the misfolded SOD1 (mSOD1) mouse model (Snow, Turnbull et al. 2003, Scott, Kranz et al. 2008), it probably would have failed preclinical testing had it been developed after hSOD1^{G93A} mice were accepted as the “gold standard”. In this study, both riluzole and edavarone were investigated together with NU-9, especially for their ability to improve axon outgrowth, branching, and arborization of diseased UMNs in culture, all accepted parameters to assess improved neuron health in vitro (Ozdinler and Macklis 2006, Jara, Genc et al. 2015, Dervishi and Ozdinler 2018).

There is an imperative need to improve the success rates of clinical trials, and that depends on our ability to obtain knowledge that directly translates to patients. UMNs in mice and UMNs in humans share many common features of motor neuron biology and display identical characteristics of neuropathology at the cellular level (Geevasinga, Menon et al. 2016, Genc, Jara et al. 2017, Jara, Genc et al. 2017, Gautam, Jara et al. 2019, Jara, Gautam et al. 2019). Therefore, information obtained directly from UMNs of well-defined mouse models of motor neuron disease at the cellular level is faithfully recapitulated in the UMNs of patients. Shifting our focus from mice to affected neurons appears to be the path forward to identify compounds that will improve the health of neurons that degenerate in patients (Genc and Ozdinler 2014, Dervishi and Ozdinler 2018, Genc, Gozutok et al. 2019) and translate from mice to humans. Furthermore, drug companies and the FDA now demand more information on the efficacy of compounds at the cellular level. We recently generated a reporter line for UMNs, UCHL1-eGFP mice, in which UMNs are genetically labeled with eGFP expression that is stable and long-lasting (Yasvoina, Genc et al. 2013). Upon crossbreeding with mouse disease models that display UMN vulnerability and progressive degeneration, such as the hSOD1^{G93A} (Gurney, Pu et al. 1994) and the TDP-43^{A315T} mice (Wegorzewska, Bell et al. 2009), we generated UMN reporter lines of disease models. The UMNs in these mouse models become diseased as a result of mSOD1 toxicity and TDP-43 pathology, two distinct causes of motor neuron death. Most importantly, because UMNs express eGFP, they can be distinguished among the thousands of other cortical cells and neurons, and their cellular responses to compound treatment can be quantitatively assessed both in vitro and in vivo (Yasvoina, Genc et al. 2013, Genc and Ozdinler 2014, Dervishi and Ozdinler 2018, Gautam, Jara et al. 2019). This represents a paradigm shift in drug discovery efforts by performing comparisons at the cellular level and utilizing the response of diseased UMNs to compound treatment as a direct readout.

Here we report the first compound, NU-9, that can improve the neuronal integrity of UMNs that become diseased by both mSOD1 toxicity and TDP-43 pathology, two nonoverlapping causes of ALS. NU-9 enhances axon outgrowth, branching, and arborization of UMNs that become diseased as a result of mSOD1 toxicity and TDP-43 pathology in vitro, better than the two FDA-approved drugs for ALS.

### Materials and Methods

NU-9. NU-9 was prepared as described in (Zhang, Benmohamed et al. 2012).

Mice. All animal procedures were approved by the Northwestern University Animal Care and Use committee and comply with the standards of the National Institutes of Health. All mice were on C57BL/6 background. Transgenic hemizygous males expressing a high copy number of the human SOD1 gene with a G93A mutation (B6SJL-Tg (SOD1*G93A)1Gur/J; The Jackson Laboratory) were bred to hemizygous UCHL1-eGFP females to generate hSOD1^{G93A}-UeGFP and WT-UeGFP (control) mice. UCHL1-eGFP mice were generated in the Ozdinler Lab; they are reporter lines for UMNs (Yasvoina, Genc et al. 2013), and are now available at Jackson Laboratory (stock no. 022476). Hemizygous UCHL1-eGFP females were bred to hemizygous prpTDP-43^{A315T} mice (procured from Jackson Laboratory, stock no. 010700) to generate prpTDP-43^{A315T}-UeGFP mice. prpTDP-43^{A315T} mice were supplied with gel diet (DietGel 76A, ClearH₂O, ME, USA) to eliminate gastrointestinal (GI) complications. Transgenic mice were identified by PCR amplification of DNA extracted from their tail, as previously described (Gurney, Pu et al. 1994, Wegorzewska, Bell et al. 2009, Yasvoina, Genc et al. 2013, Gautam, Jara et al. 2019). Mice with different genotypes are abbreviated in the figures as follows: WT-UeGFP (control) mice=G mice; hSOD1^{G93A}-UeGFP mice=GS mice; prpTDP-43^{A315T}-UeGFP mice=GTDP mice.

Compound preparation and delivery. NU-9 was prepared as 100 μM stock in dimethylsulfoxide (DMSO) and added to serum free medium (SFM) at a final concentration of 400 nM (4 μl per 1 ml SFM). Riluzole (Acros organics) was prepared as 200 μM stock in DMSO and added to SFM at a final concentration of 500 nM (2.5 μl per 1 ml SFM). Edaravone (Sigma-Aldrich) was prepared as 1 mM stock in DMSO and added at a final concentration of 1 μM in SFM (1 μl per 1 ml SFM). Amylyx AMX-0035 compound was a combination of 1 mM of 4-Phenylbutyric acid (4-PBA, Sigma Aldrich)+100 μM Tauroursodeoxycholic Acid, Sodium Salt (TUDCA, Millipore Sigma) added into SFM (4-PBA was prepared as a 21.5 mM stock in water and added at 46.5 μl per 1 ml SFM, TUDCA was prepared as 20 mM stock in water, and added at 5 μl per 1 ml SFM).

UMN Cultures. P3 motor cortices isolated from WT-UeGFP, hSOD1^{G93A}-UeGFP, and prpTDP-43^{A315T}-UeGFP mice were dissected, dissociated, and cultured on glass coverslips (4×10⁴ cells per 18 mm diameter coverslip, Fisherbrand) coated with poly-L-lysine (10 mg/mL, Sigma) as previously described (Ozdinler and Macklis 2006). Neurons were cultured in SFM [0.034 mg/L BSA, 1 mM L-glutamine, 25 U/mL penicillin, 0.025 mg/mL streptomycin, 35 mM glucose, and 0.5% B27 in Neurobasal-A medium (Life Technologies)] in a humidified tissue culture incubator in the presence of 5% CO₂ at 37° C. NU-9 (400 nM), riluzole (500 nM, Acros organics), edaravone (1 μM, Sigma-Aldrich), and AMX-0035 (1 mM 4-PBA, Sigma Aldrich+100 μM TUDCA, Millipore) were added at the start of the culture. Cultures were fixed after 3 days in vitro (DIV). These concentrations were chosen based on the concentration that were calculated and reported to be present in the CNS at their optimum dose, for each compound.

UMNs were quantitatively analyzed for differences in neurite length and arborization complexity. Images taken with a 20× objective on the epifluorescent microscope (Nikon) were analyzed using the Neurite Tracer plugin from FIJI (NIH), which enables semi-autonomous tracing to measure the length of the axon. The aggregation of the neurite tracings centered at the soma generates a profile available for Sholl analysis.

Immunocytochemistry. The antibodies used are as follows: anti-GFP (1:1000, Invitrogen; or 1:1000, Abcam). Briefly, sections were treated with blocking solution (PBS, 0.05% BSA, 2% FBS, 1% Triton X-100, and 0.1% saponin) for 30 min at room temperature and incubated with primary antibody diluted in blocking solution overnight at 4° C. Secondary fluorescent antibodies (1:500, AlexaFluor-488 conjugated, Invitrogen) were added to the blocking solution at room temperature for 2 h in the dark. Nuclei were counterstained with DAPI.

Statistical analysis. All analyses were performed using Prism software (GraphPad Software). The D'Agostino and Pearson normality test was performed on all datasets. Statistical differences between more than two groups were determined by one-way ANOVA followed by the Tukey's post-hoc multiple-comparison test. Two-way ANOVA with Sidak's multiple comparisons test was used to determine the significance of each cell mean with the other cell mean in that row for Sholl analysis results. Statistically significant differences were taken at p<0.05.

### Results

Identification of NU-9 as candidate. Repeated dose toxicity study was performed at Laboratory Animal House (LAH), Toxicology, Sai Life Science Ltd., Chrysalis Enclave, Phase II, Hinjewadi, Pune-411 057, Maharashtra, India. NU-9 when administered to male BALB/c mice, once daily for 7 consecutive days by oral (gavage) route 100 mg/kg/day dose did not result in mortality. Following repeated oral dose administration of NU-9 for 7 consecutive days in male BALB/c mice, the plasma concentrations on Day 7 were quantifiable till 24 h with Tmax 0.5 h. Brain concentrations on day 7 were quantifiable up to 24 h. When administered to mice at a dose of 100 mg/kg/day via daily gavage the dose of NU-9 detected in CNS was 400 nM. 100 mg/kg/day of NU-9 via daily gavage was determined to be the most effective dose in vivo (Genc, Gautam et al. 2021), therefore 400 nM dose was selected for in vitro experiments.

NU-9 enhances axon outgrowth of diseased UMNs. Since axonal degeneration is an important contributor to UMN loss, any effective treatment strategy will require enhancing the health and stability of UMN axon. We thus investigated whether NU-9 were capable of promoting and enhancing axon outgrowth of diseased UMNs. Because UMNs are eGFP⁺ in hSOD1^{G93A}-UeGFP mice, they can be distinguished among other cortical cells and neurons of the motor cortex in vitro. UMNs retain their pyramidal neuron shape and neuronal identity and respond to compound treatment in culture (Dervishi and Ozdinler 2018). In an effort to determine whether NU-9 improves UMN axon outgrowth and whether this is comparable to that of previously approved drugs for ALS (i.e., riluzole and edaravone), we introduced NU-9 and drugs homogeneously to the culture medium. We used the concentrations that were previously calculated and reported to be present in the CNS after administration of the optimum dose used in clinical trials for riluzole (500 nM; (Lacomblez, Bensimon et al. 1996, Scott, Kranz et al. 2008, Bellingham 2011)), edaravone (1 μM; (Ito, Wate et al. 2008)), and AMX-0035 (Paganoni, Macklin et al. 2020, Paganoni, Hendrix et al. 2021). Based on pharmacokinetic studies performed on NU-9, the dose detected in CNS was 400 nM when administered to mice at a dose of 100 mg/kg/day via daily gavage. Therefore, rather than assaying similar doses of compounds, the previously determined optimum active doses of NU-9, riluzole and edaravone in CNS were compared.

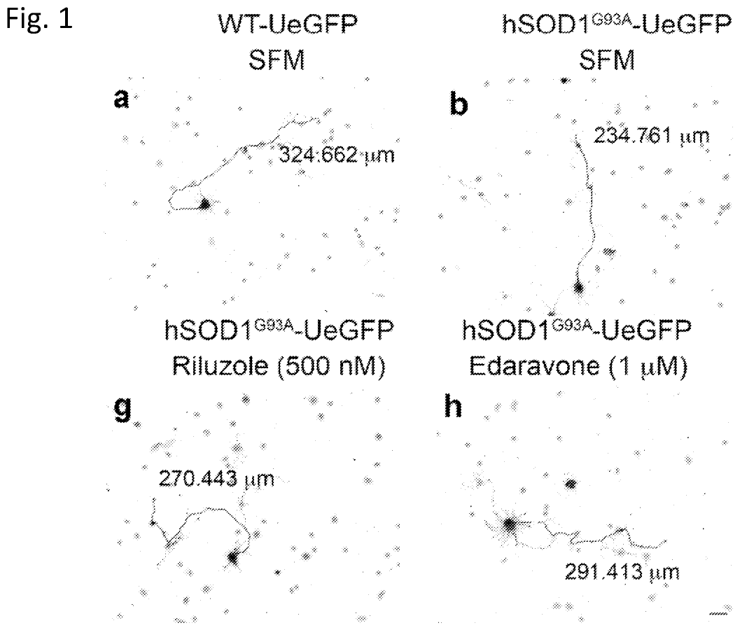

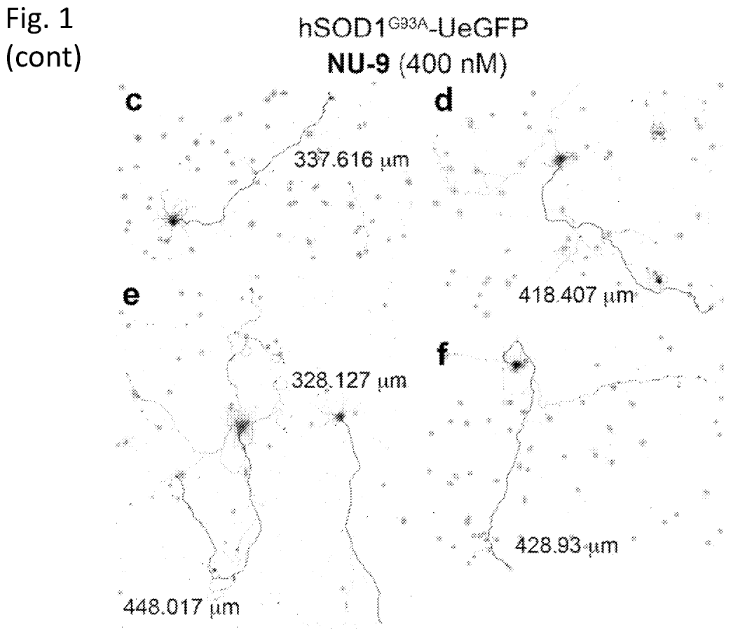

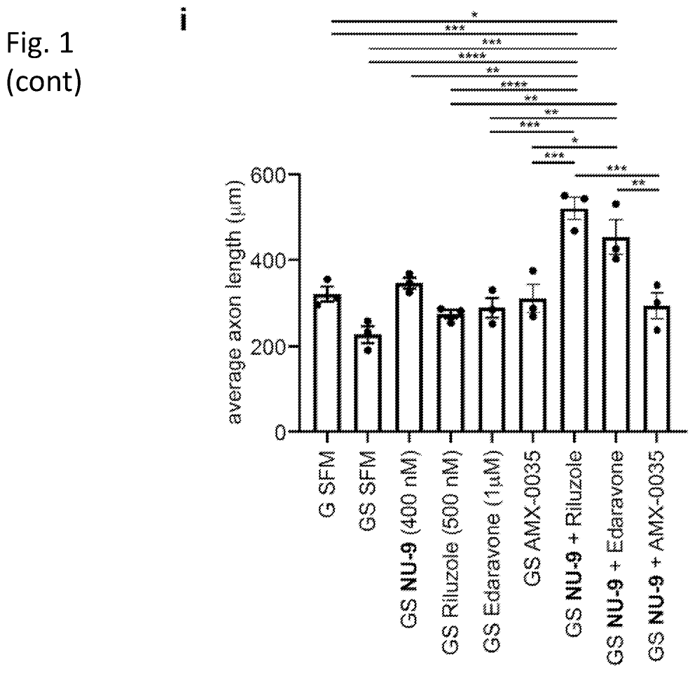

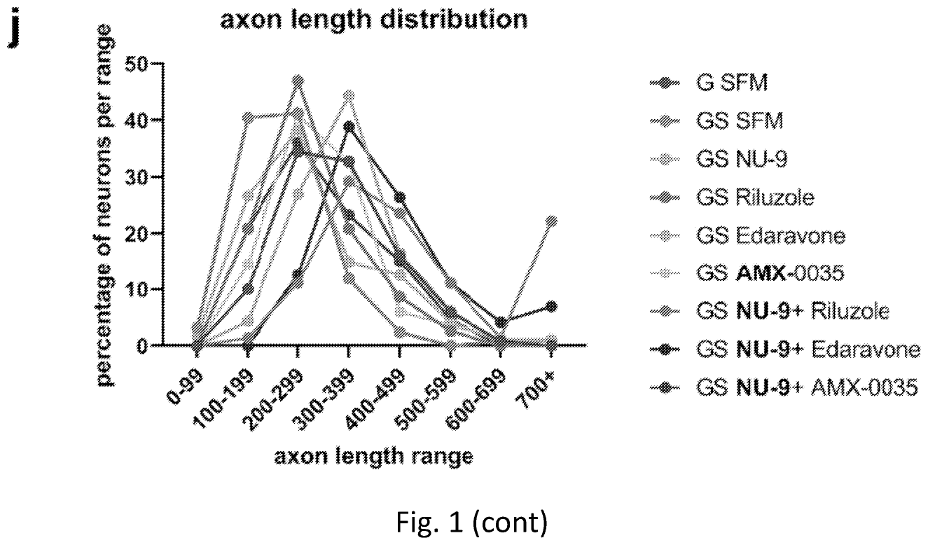

We find that UMNs of WT-UeGFP mice extend axons with an average length of 321.41±17.54 μm, (FIG. 1a), whereas diseased UMNs of hSOD1^{G93A}-UeGFP mice extend a shorter axon (227.62±19.63 μm; adjusted p-value=0.1303, FIG. 1b). However, addition of NU-9 to the culture medium significantly improves the average length of UMN axons (346.76±12.32 μm; adjusted p-value=0.0338; FIG. 1c-f). Both riluzole (500 nM) and edaravone (1 μM) also slightly increase the average length of diseased UMNs, albeit they fail to reach significance (riluzole (500 nM): 274.83±9.83 μm adjusted p-value=0.7732, FIG. 1g; edaravone (1 μM): 289.58±22.71 μm; adjusted p-value=0.5171, FIG. 1h,i). However, when NU-9 is added in combination with riluzole or edaravone, average length of the longest axon is longer than either drug alone (NU-9+riluzole: 521.18±26.78 μm adjusted p-value compared to SFM<0.0001, adjusted p-value compared to NU-9 alone=0.0016, adjusted p-value compared to riluzole alone<0.0001; NU-9+edaravone: 453.30±39.87 μm adjusted p-value compared to SFM=0.0001, adjusted p-value compared to NU-9 alone=0.067 (n.s.), adjusted p-value compared to edaravone alone=0.0029).

Plotting the distribution of longest axons revealed that UMNs of hSOD1^{G93A}-UeGFP mice cultured in SFM mostly had shorter axons (39.3% within the range of 100-199 μm and 41.8% within the range of 200-299 μm), whereas NU-9 treatment caused an overall shift, resulting in about 44.4% of UMNs to have an axon within the range of 300-399 μm. Both riluzole and edaravone improved overall axon outgrowth (FIG. 1j), but not as profoundly as NU-9.

Neuronal arborization and branching is yet another measure used to determine whether the health of the neuron is improved by compound treatment (Ozdinler, Benn et al. 2011). Sholl measurements further confirmed that NU-9 treatment resulted in the generation of more complex and arborized UMNs, even after 3 days in culture (data not shown). Both riluzole and edaravone also improved UMN arborization, but to a lesser extent (data not shown).

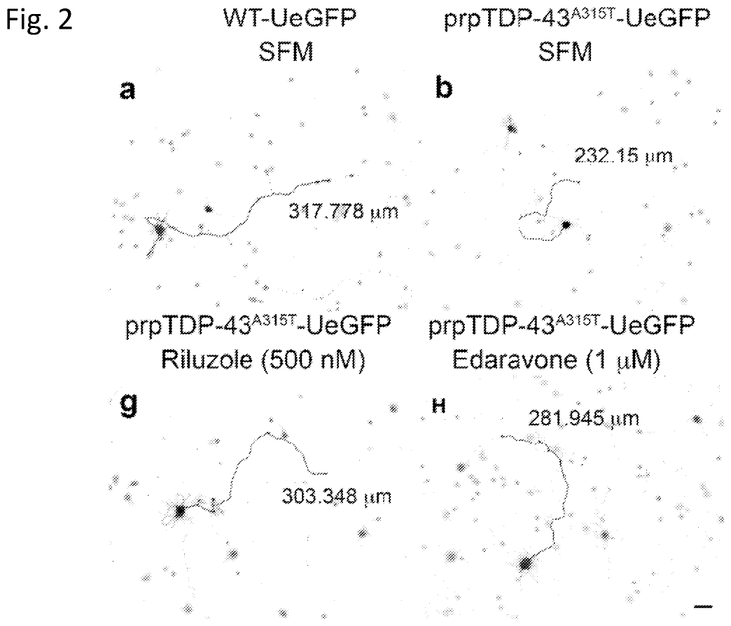

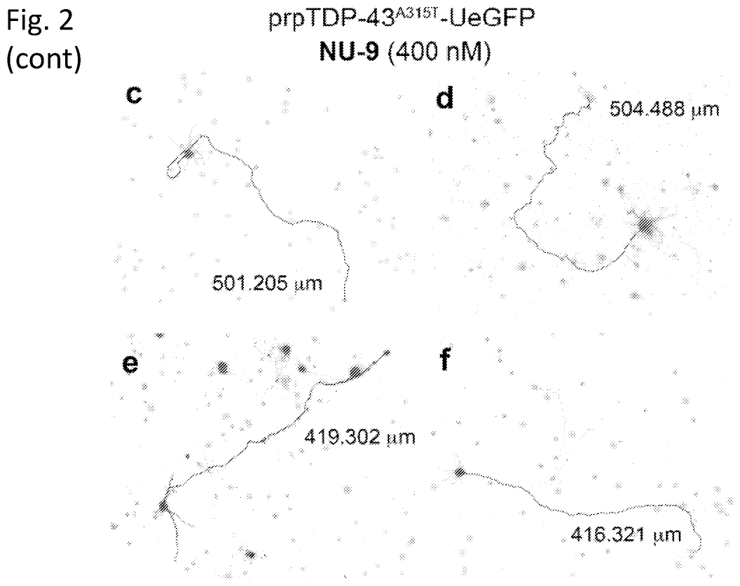

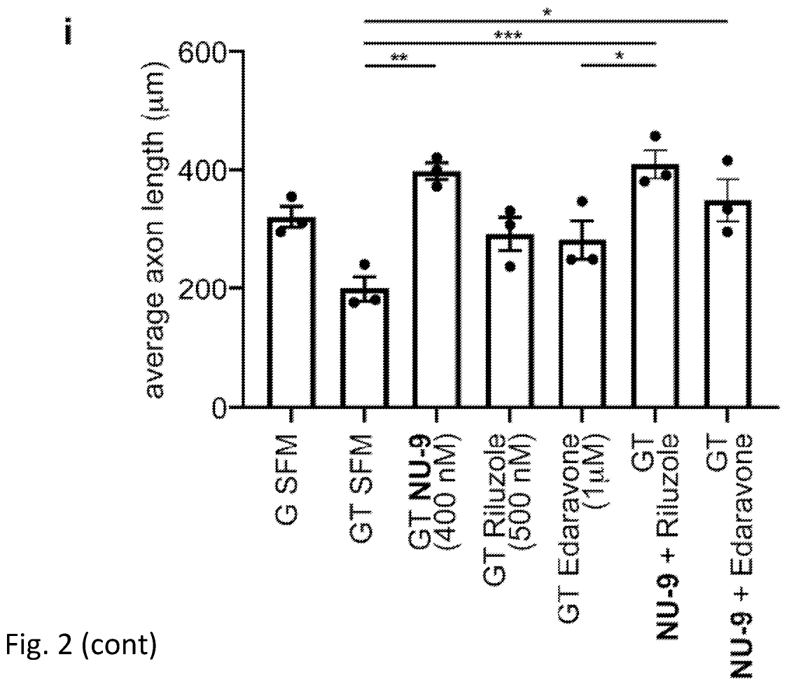

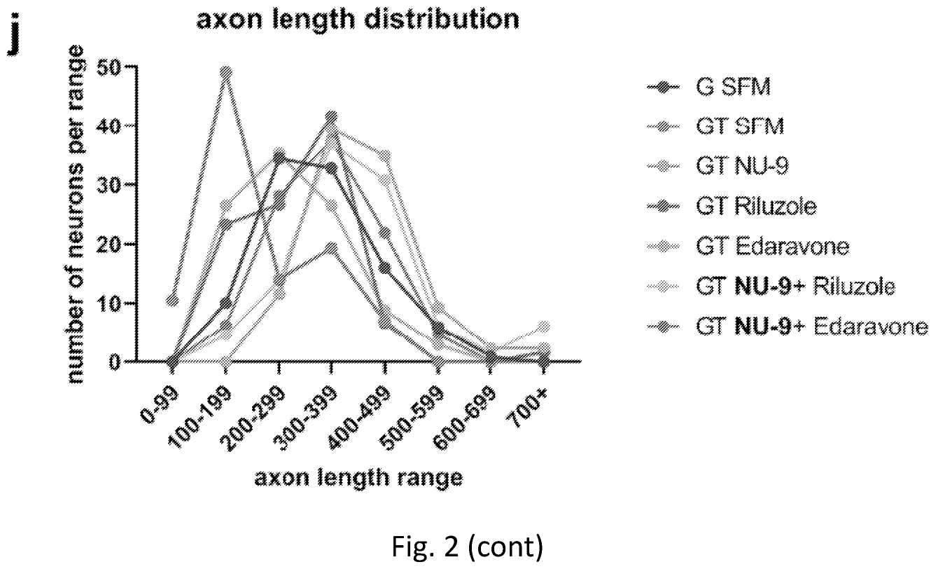

NU-9 improves axon outgrowth of UMNs with TDP-43 pathology. UMNs of WT-UeGFP mice extended axons with an average length of 321.41±17.54 μm, (FIG. 2a), whereas UMNs diseased with TDP-43 pathology had shorter axons (prpTDP-43^{A315T}-UeGFP: 199.52±21.15 μm; adjusted p-value=0.0549 (n.s.); FIG. 2b). When UMNs from prpTDP-43^{A315T}-UeGFP mice were treated with 400 nM of NU-9 in culture, the average length of the axons of UMNs significantly increased (397.73±14.09 μm; adjusted p-value=0.0013; FIG. 2c-f). Treatment with riluzole (500 nM; FIG. 2g) and edaravone (1 μM; FIG. 2h) slightly increased the average length of UMNs with TDP-43 pathology, but failed to have a significant impact (riluzole, 292.60±28.03 μm, SFM vs. riluzole adjusted p-value=0.9817 (n.s.); edaravone, 282.57±32.39 μm, SFM vs. edaravone, adjusted p-value=0.9267 (n.s.); FIG. 2i). When NU-9 is added in combination with riluzole or edaravone, average length of the longest axon is similar to either drug alone (NU-9+riluzole: 409.59±23.78 μm adjusted p-value compared to SFM=0.0007, adjusted p-value compared to NU-9 alone=0.9999, adjusted p-value compared to riluzole alone=0.0697; NU-9+edaravone: 348.83±35.31 μm adjusted p-value compared to SFM=0.014, adjusted p-value compared to NU-9 alone=0.8186 (n.s.), adjusted p-value compared to edaravone alone=0.5523 (n.s.)).

Plotting the percent distribution of neurons per axon length range further revealed a clear shift in the percentage of UMNs with axons that are longer than 300 μm, (prpTDP-43^{A315T}-UeGFP vehicle 26% of UMNs, prpTDP-43^{A315T}-UeGFP NU-9 88% of UMNs) (FIG. 2j). Even though both riluzole and edaravone improved UMN axon outgrowth, most neurons were only in the range of 200-399 μm (riluzole 68%; edaravone 62%) (FIG. 2j). Sholl analyses (data not shown) also revealed striking arborization and branching differences between healthy and diseased UMNs and revealed how NU-9 treatment enhanced neuronal arborization and branching, another indication of improved health.

### REFERENCES

Bellingham, M. C. (2011). “A review of the neural mechanisms of action and clinical efficiency of riluzole in treating amyotrophic lateral sclerosis: what have we learned in the last decade?” CNS Neurosci Ther 17(1): 4-31.
Dervishi, I. and P. H. Ozdinler (2018). “Incorporating upper motor neuron health in ALS drug discovery.” Drug Discov Today 23(3): 696-703.
Gautam, M., J. H. Jara, N. Kocak, L. E. Rylaarsdam, K. D. Kim, E. H. Bigio and P. Hande Ozdinler (2019). “Mitochondria, ER, and nuclear membrane defects reveal early mechanisms for upper motor neuron vulnerability with respect to TDP-43 pathology.” Acta Neuropathol 137(1): 47-69.
Geevasinga, N., P. Menon, P. H. Ozdinler, M. C. Kiernan and S. Vucic (2016). “Pathophysiological and diagnostic implications of cortical dysfunction in ALS.” Nat Rev Neurol 12(11): 651-661.
Genc, B., M. Gautam, O. Gozutok, I. Dervishi, S. Sanchez, G. M. Goshu, N. Kocak, E. Xie, R. B. Silverman and P. H. Ozdinler (2021). “Improving mitochondria and ER stability helps eliminate upper motor neuron degeneration that occurs due to mSOD1 toxicity and TDP-43 pathology.” Clin Transl Med 11(2): e336.
Genc, B., O. Gozutok and P. H. Ozdinler (2019). “Complexity of Generating Mouse Models to Study the Upper Motor Neurons: Let Us Shift Focus from Mice to Neurons.” Int J Mol Sci 20(16).
Genc, B., J. H. Jara, A. K. Lagrimas, P. Pytel, R. P. Roos, M. M. Mesulam, C. Geula, E. H. Bigio and P. H. Ozdinler (2017). “Apical dendrite degeneration, a novel cellular pathology for Betz cells in ALS.” Sci Rep 7:41765.
Genc, B. and P. H. Ozdinler (2014). “Moving forward in clinical trials for ALS: motor neurons lead the way please.” Drug Discov Today 19(4): 441-449.
Gurney, M. E., H. Pu, A. Y. Chiu, M. C. Dal Canto, C. Y. Polchow, D. D. Alexander, J. Caliendo, A. Hentati, Y. W. Kwon, H. X. Deng and et al. (1994). “Motor neuron degeneration in mice that express a human Cu,Zn superoxide dismutase mutation.” Science 264(5166): 1772-1775.
Homma, T., S. Kobayashi, H. Sato and J. Fujii (2019). “Edaravone, a free radical scavenger, protects against ferroptotic cell death in vitro.” Exp Cell Res 384(1): 111592.
Ikeda, K. and Y. Iwasaki (2015). “Edaravone, a Free Radical Scavenger, Delayed Symptomatic and Pathological Progression of Motor Neuron Disease in the Wobbler Mouse.” PLOS One 10(10): e0140316.
Ito, H., R. Wate, J. Zhang, S. Ohnishi, S. Kaneko, H. Ito, S. Nakano and H. Kusaka (2008). “Treatment with edaravone, initiated at symptom onset, slows motor decline and decreases SOD1 deposition in ALS mice.” Exp Neurol 213(2): 448-455.
Jara, J. H., M. Gautam, N. Kocak, E. F. Xie, Q. Mao, E. H. Bigio and P. H. Ozdinler (2019). “MCP1-CCR2 and neuroinflammation in the ALS motor cortex with TDP-43 pathology.” J Neuroinflammation 16(1): 196.
Jara, J. H., B. Genc, G. A. Cox, M. C. Bohn, R. P. Roos, J. D. Macklis, E. Ulupinar and P. H. Ozdinler (2015). “Corticospinal Motor Neurons Are Susceptible to Increased ER Stress and Display Profound Degeneration in the Absence of UCHL1 Function.” Cereb Cortex 25(11): 4259-4272.
Jara, J. H., B. Genc, M. J. Stanford, P. Pytel, R. P. Roos, S. Weintraub, M. M. Mesulam, E. H. Bigio, R. J. Miller and P. H. Ozdinler (2017). “Evidence for an early innate immune response in the motor cortex of ALS.” J Neuroinflammation 14(1): 129.
Lacomblez, L., G. Bensimon, P. N. Leigh, P. Guillet and V. Meininger (1996). “Dose-ranging study of riluzole in amyotrophic lateral sclerosis. Amyotrophic Lateral Sclerosis/Riluzole Study Group II.” Lancet 347(9013): 1425-1431.
Ozdinler, P. H., S. Benn, T. H. Yamamoto, M. Guzel, R. H. Brown, Jr. and J. D. Macklis (2011). “Corticospinal motor neurons and related subcerebral projection neurons undergo early and specific neurodegeneration in hSOD1G(9)(3) A transgenic ALS mice.” J Neurosci 31(11): 4166-4177.
Ozdinler, P. H. and J. D. Macklis (2006). “IGF-I specifically enhances axon outgrowth of corticospinal motor neurons.” Nat Neurosci 9(11): 1371-1381.
Paganoni, S., S. Hendrix, S. P. Dickson, N. Knowlton, E. A. Macklin, J. D. Berry, M. A. Elliott, S. Maiser, C. Karam, J. B. Caress, M. A. Owegi, A. Quick, J. Wymer, S. A. Goutman, D. Heitzman, T. D. Heiman-Patterson, C. E. Jackson, C. Quinn, J. D. Rothstein, E. J. Kasarskis, J. Katz, L. Jenkins, S. Ladha, T. M. Miller, S. N. Scelsa, T. H. Vu, C. N. Fournier, J. D. Glass, K. M. Johnson, A. Swenson, N. A. Goyal, G. L. Pattee, P. L. Andres, S. Babu, M. Chase, D. Dagostino, M. Hall, G. Kittle, M. Eydinov, M. McGovern, J. Ostrow, L. Pothier, R. Randall, J. M. Shefner, A. V. Sherman, M. E. St Pierre, E. Tustison, P. Vigneswaran, J. Walker, H. Yu, J. Chan, J. Wittes, Z. F. Yu, J. Cohen, J. Klee, K. Leslie, R. E. Tanzi, W. Gilbert, P. D. Yeramian, D. Schoenfeld and M. E. Cudkowicz (2021). “Long-term survival of participants in the CENTAUR trial of sodium phenylbutyrate-taurursodiol in amyotrophic lateral sclerosis.” Muscle Nerve 63(1): 31-39.
Paganoni, S., E. A. Macklin, S. Hendrix, J. D. Berry, M. A. Elliott, S. Maiser, C. Karam, J. B. Caress, M. A. Owegi, A. Quick, J. Wymer, S. A. Goutman, D. Heitzman, T. Heiman-Patterson, C. E. Jackson, C. Quinn, J. D. Rothstein, E. J. Kasarskis, J. Katz, L. Jenkins, S. Ladha, T. M. Miller, S. N. Scelsa, T. H. Vu, C. N. Fournier, J. D. Glass, K. M. Johnson, A. Swenson, N. A. Goyal, G. L. Pattee, P. L. Andres, S. Babu, M. Chase, D. Dagostino, S. P. Dickson, N. Ellison, M. Hall, K. Hendrix, G. Kittle, M. McGovern, J. Ostrow, L. Pothier, R. Randall, J. M. Shefner, A. V. Sherman, E. Tustison, P. Vigneswaran, J. Walker, H. Yu, J. Chan, J. Wittes, J. Cohen, J. Klee, K. Leslie, R. E. Tanzi, W. Gilbert, P. D. Yeramian, D. Schoenfeld and M. E. Cudkowicz (2020). “Trial of Sodium Phenylbutyrate-Taurursodiol for Amyotrophic Lateral Sclerosis.” N Engl J Med 383(10): 919-930.
Scott, S., J. E. Kranz, J. Cole, J. M. Lincecum, K. Thompson, N. Kelly, A. Bostrom, J. Theodoss, B. M. Al-Nakhala, F. G. Vieira, J. Ramasubbu and J. A. Heywood (2008). “Design, power, and interpretation of studies in the standard murine model of ALS.” Amyotroph Lateral Scler 9(1): 4-15.
Snow, R. J., J. Turnbull, S. da Silva, F. Jiang and M. A. Tarnopolsky (2003). “Creatine supplementation and riluzole treatment provide similar beneficial effects in copper, zinc superoxide dismutase (G93A) transgenic mice.” Neuroscience 119(3): 661-667.
Wegorzewska, I., S. Bell, N. J. Cairns, T. M. Miller and R. H. Baloh (2009). “TDP-43 mutant transgenic mice develop features of ALS and frontotemporal lobar degeneration.” Proc Natl Acad Sci USA 106(44): 18809-18814.
Writing, G. and A. L. S. S. G. Edaravone (2017). “Safety and efficacy of edaravone in well defined patients with amyotrophic lateral sclerosis: a randomised, double-blind, placebo-controlled trial.” Lancet Neurol 16(7): 505-512.
Yasvoina, M. V., B. Genc, J. H. Jara, P. L. Sheets, K. A. Quinlan, A. Milosevic, G. M. Shepherd, C. J. Heckman and P. H. Ozdinler (2013). “eGFP expression under UCHL1 promoter genetically labels corticospinal motor neurons and a subpopulation of degeneration-resistant spinal motor neurons in an ALS mouse model.” J Neurosci 33(18): 7890-7904.
Yoshida, H., A. H. Kwon, K. Habara, M. Yamada, M. Kaibori, Y. Kamiyama, M. Nishizawa, S. Ito and T. Okumura (2008). “Edaravone inhibits the induction of iNOS gene expression at transcriptional and posttranscriptional steps in murine macrophages.” Shock 30(6): 734-739.
Yoshida, H., A. H. Kwon, M. Kaibori, K. Tsuji, K. Habara, M. Yamada, Y. Kamiyama, M. Nishizawa, S. Ito and T. Okumura (2008). “Edaravone prevents iNOS expression by inhibiting its promoter transactivation and mRNA stability in cytokine-stimulated hepatocytes.” Nitric Oxide 18(2): 105-112.
Yoshino, H. and A. Kimura (2006). “Investigation of the therapeutic effects of edaravone, a free radical scavenger, on amyotrophic lateral sclerosis (Phase II study).” Amyotroph Lateral Scler 7(4): 241-245.
Zhang, Y., R. Benmohamed, W. Zhang, J. Kim, C. K. Edgerly, Y. Zhu, R. I. Morimoto, R. J. Ferrante, D. R. Kirsch and R. B. Silverman (2012). “Chiral cyclohexane 1,3-diones as inhibitors of mutant SOD1-dependent protein aggregation for the treatment of ALS.” ACS Med Chem Lett 3(7): 584-587.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

1. A method for treating amyotrophic lateral sclerosis or symptoms thereof in a subject in need thereof, the method comprising administering to the subject an effective amount of (S)-5-(1-(3,5-bis(trifluoromethyl)phenoxy)ethyl)cyclohexane-1,3-dione, or a pharmaceutically acceptable salt thereof, to improve health of diseased upper motor neurons.

2. The method of claim 1, further comprising administering to the subject an effective amount of riluzole, or a pharmaceutically acceptable salt thereof.

3. The method of claim 1, further comprising administering to the subject an effective amount of edaravone, or a pharmaceutically acceptable salt thereof.

4. The method of claim 1, wherein the effective amount of (S)-5-(1-(3,5-bis(trifluoromethyl)phenoxy)ethyl)cyclohexane-1,3-dione, or a pharmaceutically acceptable salt thereof, is administered before, concurrently with, or after administering to the subject an effective amount of riluzole, edaravone, or a pharmaceutically acceptable salt thereof.

5. The method of claim 1, wherein symptoms of ALS comprise diseased upper motor neurons in the subject.

6. A method for improving the health of diseased upper motor neurons in a subject in need thereof, the method comprising administering to the subject an effective amount of (S)-5-(1-(3,5-bis(trifluoromethyl)phenoxy)ethyl)cyclohexane-1,3-dione, or a pharmaceutically acceptable salt thereof.

7. The method of claim 6, further comprising administering to the subject an effective amount of riluzole, or a pharmaceutically acceptable salt thereof.

8. The method of claim 6, further comprising administering to the subject an effective amount of edaravone, or a pharmaceutically acceptable salt thereof.

9. The method of claim 6, wherein the effective amount of (S)-5-(1-(3,5-bis(trifluoromethyl)phenoxy)ethyl)cyclohexane-1,3-dione, or a pharmaceutically acceptable salt thereof, is administered before, concurrently with, or after administering to the subject an effective amount of riluzole, edaravone, or a pharmaceutically acceptable salt thereof.

10. The method of claim 6, wherein the subject has amyotrophic lateral sclerosis or symptoms thereof.

11. A unit dosage package for use in the method of claim 1 comprising:
(i) (S)-5-(1-(3,5-bis(trifluoromethyl)phenoxy)ethyl)cyclohexane-1,3-dione, or a pharmaceutically acceptable salt thereof; and
(ii) riluzole, or a pharmaceutically acceptable salt thereof, or edaravone, or a pharmaceutically acceptable salt thereof.

12. The unit dosage package of claim 11 comprising riluzole, or a pharmaceutically acceptable salt thereof.

13. The unit dosage package of claim 11 comprising edaravone, or a pharmaceutically acceptable salt thereof.

14. A pharmaceutical composition for use in the method of claim 1 comprising:
(i) (S)-5-(1-(3,5-bis(trifluoromethyl)phenoxy)ethyl)cyclohexane-1,3-dione, or a pharmaceutically acceptable salt thereof;
(ii) riluzole, or a pharmaceutically acceptable salt thereof, or edaravone, or a pharmaceutically acceptable salt thereof; and
(iii) a pharmaceutically acceptable carrier or excipient.

15. The pharmaceutical composition of claim 14 comprising riluzole, or a pharmaceutically acceptable salt thereof.

16. The pharmaceutical composition of claim 14 comprising edaravone, or a pharmaceutically acceptable salt thereof.

17. A method for detecting candidate compounds that improve health of diseased upper motor neurons comprising:
(i) culturing diseased upper motor neurons in the presence and absence of a candidate compound;
(ii) detecting one or more parameters related to upper motor neuron health in the cells of step (i);
(iii) generating a test index by calculating a change in the one or more parameters between the diseased upper motor neurons cultured in the presence and absence of the candidate compound and generating a control index by calculating a change in the one or more parameters between the cells cultured in the presence and absence of a control substance;
wherein, if the value of the test index is greater than, or improved, as compared to the value of the control index, then the candidate compound improves the health of diseased upper motor neurons.

18. The method of claim 17, wherein the diseased upper motor neurons become diseased by mSOD1 toxicity or TDP-43 pathology.

19. (canceled)

20. The method of claim 17, wherein the one or more parameters between the diseased upper motor neurons cultured in the presence and absence of the candidate compound is axon length, neuronal arborization, or neuronal branching.

21. (canceled)

22. (canceled)

23. The method of claim 17, wherein the control substance comprises riluzole, edaravone, AMX-0035, or (S)-5-(1-(3,5-bis(trifluoromethyl)phenoxy)ethyl)cyclohexane-1,3-dione.

24-27. (canceled)
